(19) United States
(12) Patent Application Publication
(10) Pub. No.: US 2025/0302569 A1
(43) Pub. Date: Oct. 2, 2025

(54) APPARATUS

(71) Applicant: JB Surgical Pty Ltd, South Australia (AU)

(72) Inventors: Benjamin Paul LITTLEJOHNS, South Australia (AU); Jacob Andrew CHISHOLM, South Australia (AU)

(21) Appl. No.: 18/872,786

(22) PCT Filed: Jun. 7, 2022
(86) PCT No.: PCT/AU2022/050555

§ 371 Date: Dec. 6, 2024

(30) Foreign Application Priority Data
Jun. 7, 2021 (AU) 2021901689

(51) Int. Cl. A61B 50/22; A61B 17/00; A61B 46/23

(52) U.S. Cl. CPC A61B 50/22; A61B 46/23; A61B 17/00234

(57) ABSTRACT

An apparatus including:
one or more instrument ports configured to hold respectively one or more laparoscopic instruments during surgery,
wherein a top opening of each instrument port includes a downward groove configured to receive and hold a handle of a laparoscopic hand instrument resting in the port such that the handle is oriented towards a surgeon during use.

### RELATED APPLICATION

The present patent application is related to Australian Provisional Patent Application No. 2021901689, the originally filed specification of which is hereby incorporated by reference herein.

### TECHNICAL FIELD

The present disclosure relates to an apparatus (including an “instrument holder” or “quiver”) for holding laparoscopic instruments during surgery.

### BACKGROUND

Laparoscopic instruments include laparoscopic hand instruments and laparoscopic energy instruments or suction/irrigation devices that are used by a surgeon during surgery. The laparoscopic instruments may also be referred to as “tools”. The laparoscopic hand instruments may be referred to as “hand instruments” or “lap instruments”. Each hand instrument includes 3 to 5 hand instrument pieces that are typically assembled after sterilisation and prior to surgery. The hand instrument pieces generally include: a tip with jaws in the form of graspers or dissectors at a distal end of the instrument that are configured to operate in the patient's body; an outer shaft that penetrated the body; an inner shaft connected to the jaws that moves longitudinally; a handle at a proximal end of the instrument for the surgeon to hold the instrument, and to open/close graspers or dissectors; and a rotation wheel located where the handle meets the outer shaft (the rotation wheel, which is part of the outer shaft, rotates the inner shaft and the jaws when rotated, typically by the surgeon's index finger); and often an electrode (or “post”) projecting from the outer shaft to allow connection of an electrical current supply if required by the surgery. The handle (also referred to as a “grasping mechanism”) is similar to a pair of scissors although the shaft of the instrument is at 120 degrees to the longitudinal axis of the handle. The laparoscopic energy instruments may be referred to as “energy instruments” or “energy devices”, are used for cutting and sealing tissue, and have a handle that is around four times bigger than a hand instrument handle, and can often include a large housing and a power cord/cable connected to an external power source. Laparoscopic surgery can require suction provided by the suction/irrigation devices, e.g., an irrigation apparatus that is tube-like in appearance and has pipes/hoses that hang from the top and out the back, and connect to an air/water supply. The cord/cable or pipe/hose extend away from the handle of the instrument/device when in use.

During laparoscopic operations (“surgery”), the instruments are used inside a sterile field. The sterile field is the area around the surgical site that is considered sterile. The sterile field generally extends from the surgeon's lower waist up to their shoulders and the operating table height is adjusted accordingly. All people (surgeons, patients, and nurses) wear sterile gloves and gowns, face masks and/or drapes, and any items are covered by surgical drapes or sterilized in a steriliser (such as the hand instruments).

During surgery, the surgeon utilizes many different elongated instruments, and the surgeon will swap one instrument for another during the operation to perform different functions (this is referred to as an “instrument exchange”, i.e., when the surgeon needs to stop operating, remove the tool they are using, put it down and pick up then insert the tool they now need). During surgery, a tray or a laparoscopic quiver may be provided to hold the instruments (hand instruments and energy instruments or suction/irrigation devices) in a sterile manner where the surgeon can to pick them up and put them down.

However, for some surgical procedures, where more than one or two instruments are required, and/or when there are more instrument exchanges (when the surgeon needs to stop operating, remove the instrument they are using, put it down and pick up then insert the instruments they now need), known instrument holders may make it difficult for the surgeon to quickly and/or intuitively retrieve the desired instruments.

It is desired to address or ameliorate one or more disadvantages or limitations associated with the prior art, or to at least provide a useful alternative.

### SUMMARY

Disclosed herein is an apparatus (“laparoscopic instrument holder”) including:
one or more instrument ports configured to hold respectively (i.e., one each) one or more laparoscopic instruments during surgery,
wherein a top opening of each instrument port includes a downward groove (also referred to as a “gutter” or a “notch”) configured to receive and hold a handle of a laparoscopic hand instrument resting in the port (due to gravity, i.e., gravity pushes the handle down into the groove and resists the handle moving out) such that the handle is oriented towards a surgeon (at least not away from the surgeon) during use (because the orientation of the handle is defined by a location of the groove around the top opening).

The one or more instrument ports may include a plurality of ports, and the grooves of adjacent ports are oriented around their respective top openings such that handles of adjacent hand instruments, when in the adjacent ports, are guided away from overlapping each other (thus the handles are mutually aligned by the grooves and separated by the dividing walls when in adjacent ports).

The groove may include two edges (or faces) having opposite slopes meeting at a low point or line of the groove when in use such that the handle is urged towards the low point/line by gravity, thus orienting the handle toward the surgeon. The groove may be a V groove formed of two straight edges or a U groove formed of curved edges. At least one of the instrument ports may include two downward grooves configured to receive and hold a handle of a laparoscopic hand instrument resting in the port, including the downward groove in a front wall and a further downward groove in a side wall of the port

Disclosed herein is an apparatus (“laparoscopic instrument holder”) including:
a. one or more instrument ports configured to hold respectively (i.e., one each) one or more laparoscopic instruments during surgery; and
b. a leg curve projecting from top openings of the ports away from the surgeon when in use (in a second direction) and around/over/towards a leg of a patient. (The leg curve may keep the holder steady because it rests partially on the patient rather than just hanging from a sterile drape (also referred to as a “surgical drape”) that is placed over an upper leg of a patient.)

The leg curve includes a plurality of fastening points configured to be attached to the patient, typically to a sterile drape fastened over the patient. The fastening points include holes through the leg curve that are configured to receive clips (e.g., towel clips or surgical clips) to fasten the leg curve to the sterile drape.

The leg curve includes a curvature that substantially matches a curvature of an adult thigh, e.g., an average adult thigh or a large adult thigh, e.g., typical for a bariatric or gynaecological surgery patient.

The leg curve is rigid, including formed of hard plastic.

The leg curve may project from the top opening, and/or from a substantially straight back wall of the ports, around at least one quarter of a circle, or at least one eighth of a circle, or at least one sixteenth of a circle. The leg curve may include an average radius of between 4 cm and 20 cm, including between 8 cm and 9 cm.

The one or more instrument ports may include a D-shaped port (“D port”) configured to hold a laparoscopic energy instrument or suction/irrigation device with a power cord/cable and/or pipe/hose during surgery.

The top opening of the D port includes: a substantially straight edge and a substantially curved edge that cooperates with the straight edge to form a “D” shape. Unlike the instrument ports, the D port has a substantially flat top opening to support the handle of the laparoscopic energy instrument or the suction/irrigation device while allowing the handle of the laparoscopic energy instrument and/or the suction/irrigation device to rotate around the curved edge, e.g., as the power cord/cable and/or pipe/hose is moved during surgery (i.e., no V groove, so energy instrument and/or suction/irrigation device is free to move). In other words, the D port tube has a flat side for facing the end port (and the other ports), and an opposite curved side that forms a D-shaped horizontal cross section.

The D port and an end (terminal) one of the instrument ports may be configured to connect at least at their top openings to resist a surgeon manually inserting a laparoscopic instrument between the D port and the end during use (i.e., the D port is fastenable to the hand instrument ports). The end port may have an end (terminal) wall shaped to receive a cooperating wall of the D port to align the D port adjacent to the end port. The end wall may be substantially flat and the cooperating wall may be correspondingly and substantially flat. The D port and the end wall may include cooperating fastening elements to fasten the D port to the end port. The cooperating fastening elements may include slots in the end wall to receive cooperating projections (“studs”) of the D port. The cooperating fastening elements may include slots in the D port to receive cooperating projections (“studs”) of the end wall. The slots may include respective wide upper holes and narrow lower holes, and the cooperating projections may include stems that fit through the narrow lower holes and heads that fit through the wide upper holes but not the narrow lower holes.

Disclosed herein is an apparatus (“laparoscopic instrument holder”) including:
one or more instrument ports configured to hold respectively (i.e., one each) one or more laparoscopic instruments during surgery,
wherein a top opening of each instrument port includes at least one angled guide to guide a distal end of the instrument into a throat of the port when the instrument is being inserted into the port during use.

The at least one angled guide provides an inward tapering opening to the port, making it easier for the surgeon to manually insert the instrument into the port.

The at least one angled guide may include:
a. a shelf projecting from the top opening towards the surgeon when in use (in a first direction); and/or
b. a leg curve projecting from the top opening away from the surgeon when in use (in a second direction) and around/over/towards a leg of a patient.

Disclosed herein is an apparatus (including a “laparoscopic instrument holder”) including:
two or more instrument ports configured to hold respectively (i.e., one each) two or more laparoscopic instruments during surgery,
wherein adjacent ones of the ports are connected at least at their top openings to resist a surgeon manually inserting a laparoscopic instrument between the adjacent ports during use.

The adjacent ones of the ports form one or more pairs of adjacent ports. The adjacent ports in each pair are separated by a separating wall (“dividing wall”) that provides a wall for both of the adjacent ports.

Disclosed herein is an apparatus (“laparoscopic instrument holder”) including:
one or more instrument ports configured to hold respectively (i.e., one each) one or more laparoscopic instruments during surgery,
wherein each instrument port includes a window of transparent, waterproof material configured to display a distal end of the instrument in the port (including a tip of the instrument) to a surgeon (to allow the surgeon to see the tip of the instrument when the instrument is resting in the port).

The window may be provided by transparent, waterproof walls of the ports.

The window may extend from the bottom of the port on the same side as the deck so the surgeon can see the instrument tips during use.

The shelf includes the groove.

Each port includes walls, including lower walls formed of water-proof material and a rigid material, e.g., a hard plastic. Each port includes a lower portion that is enclosed by the lower walls to hold a distal end of the instrument including the tip in a sterile manner. The lower portion includes a sealed bottom. Each port has a length, extending from its top opening to its sealed bottom, that is selected based on expected lengths of the instruments, configured to hold hand instruments with lengths of substantially 250 mm, 330 mm and/or 430 mm.

Each port has a throat that swallows a rotation wheel of the hand instrument such that the rotation wheel falls below the top opening when being inserted into the port. The throat is fractionally larger (e.g., 10% to 20% larger) in cross section than the rotation wheel such that the rotation wheel is held (restrained) laterally by upper walls of the port when in the holder, e.g., for wheels with radii of substantially 40 to 50 mm.

Each port has a rectangular cross section such that it spans further from the patient than parallel to a longitudinal axis of the patient when in use.

The walls are substantially vertical when in use, including an inward taper from the top opening to the bottom.

The one or more instrument ports may include disposable materials, e.g., Polypropylene (PP) or polycarbonate.

The one or more instrument ports may include injection moulded plastic, e.g., Polypropylene (PP) or polycarbonate.

The one or more instrument ports may include 2, 3, 4 or 5 hand instrument ports.

### BRIEF DESCRIPTION OF THE DRAWINGS

Some embodiments of the present invention are hereinafter described, by way of non-limiting example only, including with reference to the accompanying drawings, in which some exemplary dimensions are provided in millimetres (mm) and:

FIG. 1 is a upper front right perspective view of an apparatus (“laparoscopic instrument holder”) configured to hold one or more hand instruments;

FIG. 2 is a front view of the holder;

FIG. 2A is a front view of a portion of the holder marked with the circle “A” in FIG. 2;

FIG. 3 is a right side view of the holder;

FIG. 4 is an upper front left perspective view of the holder including a D port configured to hold an energy instrument or suction/irrigation device;

FIG. 5 is a right side view of a portion of the holder marked with the circle “B” in FIG. 3;

FIG. 6 is a cross sectional right side view of the cross section marked “C”-“C” in FIG. 2;

FIG. 7 is a left side view of the holder;

FIG. 8 is a top view of the holder;

FIG. 9 is a top view of the D port; and

FIG. 10 is a perspective view of a variant of the D port with a leg curve.

### DETAILED DESCRIPTION

The apparatus (“laparoscopic instrument holder”) described herein facilitates the storage of laparoscopic surgical instruments ready for use during an operation (“surgery”), i.e., in a convenient location that is easily accessible for both the surgeon and the scrub nurse. The holder includes ergonomics that assists with presentation and delivery of the instruments to the surgeon during the surgery.

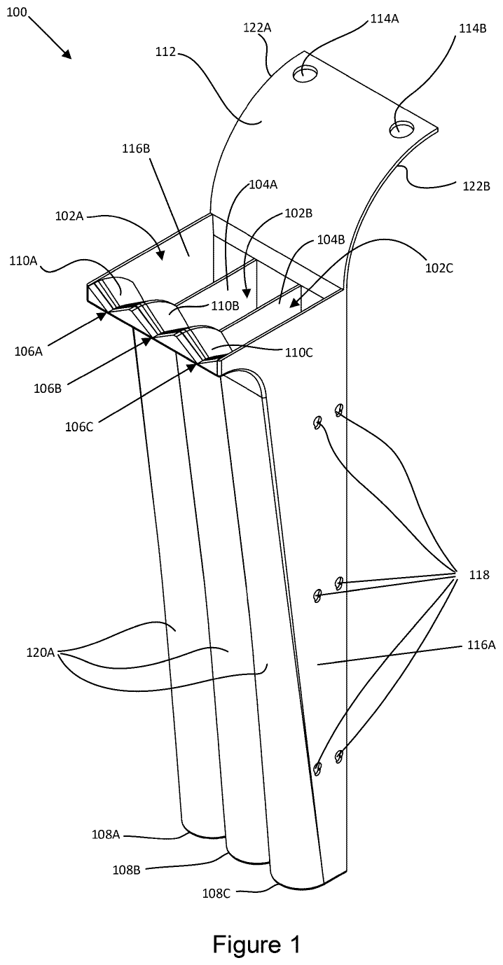

The holder 100 includes one or two or more instrument ports 102, e.g., three instruments ports 102 as shown in FIG. 1. The holder 100 may include 2, 3, 4 or 5 hand instrument ports. In preparation for surgery, the surgeon will often pick 2, 3, 4 or 5 of hand instruments, and an energy instrument or suction/irrigation device, depending on the procedure and the surgeon.

Each port 102 is configured to hold a laparoscopic hand instrument, allowing convenient manual insertion and withdrawal of the instrument into and from the port 102 during surgery. In use, the holder 100 includes up to one hand instrument in each port 102.

Adjacent ones of the ports 102 (e.g., adjacent ports 102A and 102B, or adjacent ports 102B and 102C, as shown in FIG. 1) are connected at least at their top openings to resist a surgeon manually (and erroneously, i.e., by mistake) inserting a laparoscopic instrument between the adjacent ports (e.g., between 102A and 102B, or between 102A and 102B) during use. The top openings are connected by a connection, e.g., a shared edge, between the adjacent top openings. Having such connected adjacent ports 102 may be more convenient and reliable compared to having wholly separated ports, e.g., a collection of unconnected single-instrument holders.

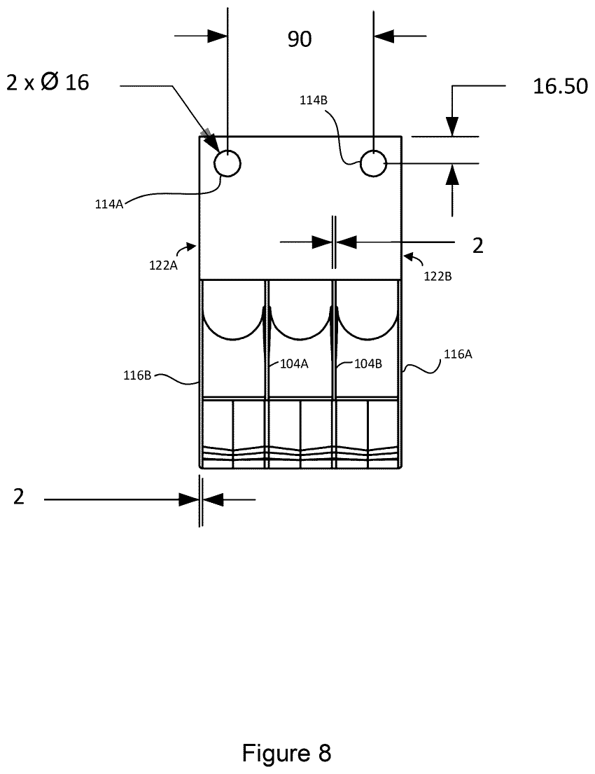

The adjacent ones of the ports 102 form one or more pairs of adjacent ports 102. The adjacent ports 102 in each pair are separated by a separating wall (or “dividing wall”) 104 that provides the shared edge and a side wall for both of the adjacent ports: e.g., as shown in FIG. 1, a dividing wall 104A is shared by and separates ports 102A and 102B, and similarly a dividing wall 104B is shared by and separates ports 102B and 102C. The dividing walls 104 act as dividers that separate the hand instruments in the holder 100 to resist mutual entanglement of the instruments. As shown in FIG. 8, the dividing walls 104 can have thicknesses of between 1 and 3 mm, e.g., substantially 2 mm.

The top opening of each port 102 includes a downward groove 106 (or “gutter” or “notch”) configured to receive and hold a handle of a laparoscopic hand instrument resting in the corresponding port 102: e.g., as shown in FIG. 1, the port 102A includes a groove 106A, the port 102B includes a groove 106B, and the port 102C includes a groove 106C. When the hand instrument is in the port 102, it is held there by gravity because the holder 102 is mounted/positioned/oriented substantially vertically (as shown in FIG. 1) with the groove 106 at the top opening facing upwards, and a bottom 108 facing downwards: gravity pushes the handle down into the groove 106 and resists the handle moving out because the groove 106 includes a centre line, and a pair of upward sloping walls that slope upward on both sides of the centre line.

The groove 106 is configured and oriented such that the handle is oriented towards the surgeon by gravity forcing the handle towards the centre line (i.e., where the surgeon generally stands, next to the holder 100 and on an opposite side of the holder 100 from the patient), or at least not away from the surgeon, during use. The orientation of the groove 106, and thus the handle, is defined by a location of the centre line of the groove 106 around the top opening: as shown in FIG. 1, the groove 106 is oriented towards where the surgeon generally stands during use (“towards the surgeon”). The groove 106 can improve the holder 100 compared to having a straight-edged top opening (without groove) because (i) the handle is naturally oriented and aligned as it falls into the groove 106, even if it is dropped/placed by the surgeon at another angle within the width of the groove 106; and (ii) some hand instruments include electrode post that projects upwards when the handle is projecting downwards that may to align itself with a corner of a rectangular port (the corner provided by a join of adjacent side walls of the port), thus tending to orient the handle at around 45 degrees to the rectangular slot, i.e., along a diagonal of the slot, thus around 45 degrees away from being towards the surgeon, thus in an overlapping position with an adjacent handle, especially if the adjacent handle is oriented towards the first handle, e.g., at 45 degrees in the opposite direction.

The grooves 106 of adjacent ports 102 (when there are multiple ports) are oriented around their respective top openings such that handles of adjacent hand instruments, when in the adjacent ports 102, are guided away from overlapping each other, thus the handles are mutually aligned and separated when in adjacent ports 102. As shown in FIG. 1, the grooves 106A, 106B and 106C are oriented in the same direction, i.e., substantially parallel, and separated by side-to-side widths of the ports since the grooves 106A, 106B and 106C are substantially central in the side-to-side width of each corresponding port 102A, 102B, 102C.

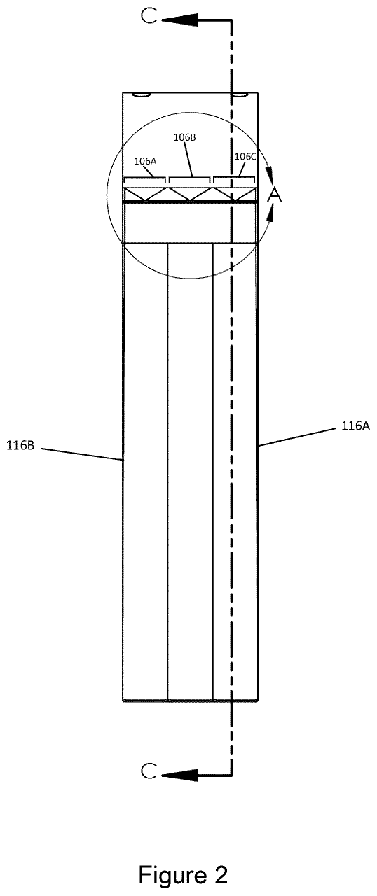

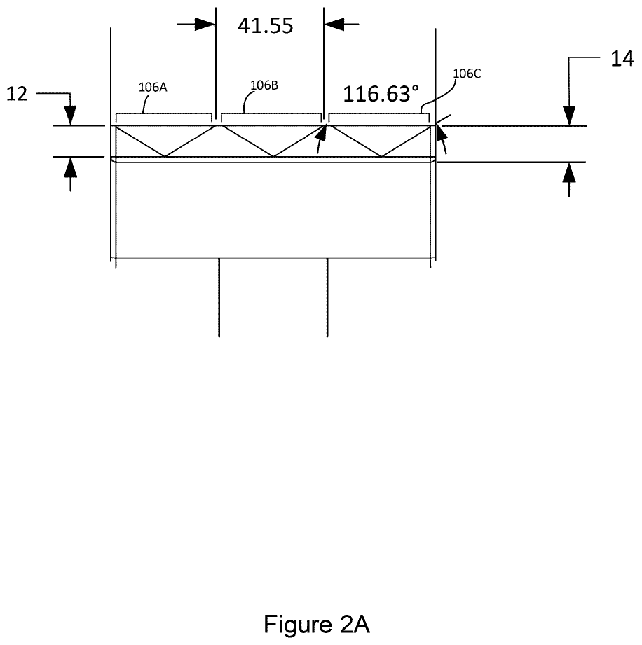

The groove 106 includes the upward sloping walls that respectively provide two edges or faces meeting at the centre line, which provides a low point or line of the groove 106 when in use such that the handle is urged towards the low point/line by gravity, thus orienting the handle toward the surgeon. The groove may be a V groove formed of two substantially straight edges, or a U groove formed of curved edges. As shown in FIGS. 2 and 2A, the grooves 106A, 106B and 106C may be V grooves, centred on the side-to-side width of each port 102A, 102B, 102C, each with two linear edges forming a symmetrical groove with a groove depth between 6 and 24 mm (or between 10 and 14 mm) and a groove width of between 20 and 80 mm (or between 40 and 44 mm), thus each linear edge forming an angle of substantially between 100 degrees and 130 degrees (or between 113 degrees and 119 degrees) from the horizontal when in use.

Each port 102 includes walls, including lower walls formed of water-proof material and a rigid material, e.g., a hard plastic, e.g., Polypropylene or polycarbonate suitable injection molding. Each port 102 includes a lower portion that is enclosed by the lower walls to hold a distal end of the instrument including the tip in a sterile manner. The lower portion includes a sealed bottom 108.

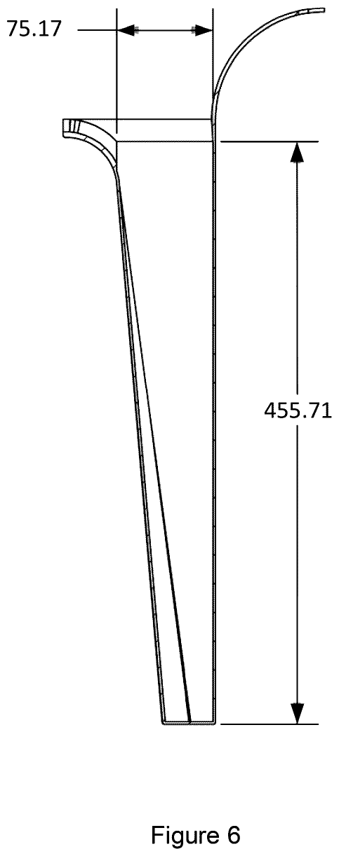

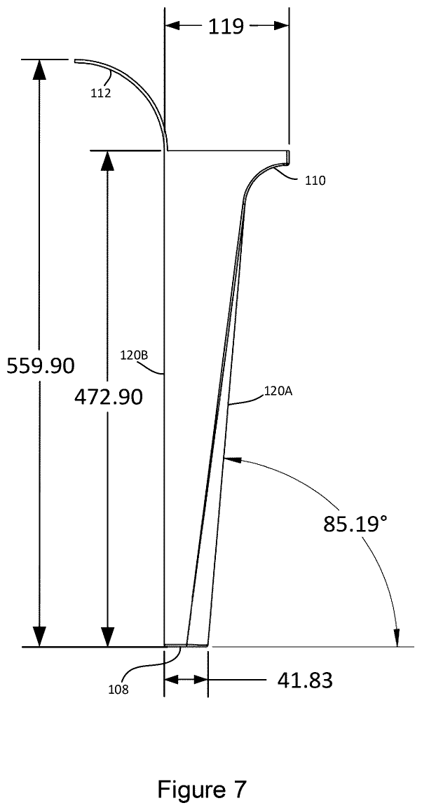

Each port 102 has a length, extending from its top opening to its sealed bottom, that is selected based on expected lengths of the instruments such that port 102 is sufficiently deep to allow the handle to rest on the top opening (i.e., so the instrument is supported by the handle on the top opening rather than the top reaching the bottom 108). The length of the port 102, i.e., in a substantially vertical direction when in use, can be between 45 cm and 50 cm, as shown in FIG. 6. The holder length (i.e., height when in use) includes the height of a shelf 110 and the leg curve 112 as shown in FIG. 7: the height of the holder 100 including the leg curve 112 can be between 27 and 60 cm (or between 55 and 57 cm), and the height of the holder 100 including the shelf 110 can be between 45 and 50 cm (or between 46 and 48 cm), as shown in FIG. 7. The height of the holder 100 including the leg curve 112 can be less than 60 cm or 70 cm for use with adult patients.

Instrument lengths are measured by the outer shaft length including 250 mm, 330 mm (most common) and 430 mm for large bariatric patients. The holder 100 is configured to receive not only the shaft length but the rotation wheel and jaw. The jaw sizes range from 30 mm to 55 mm in length plus the rotation wheel is about 60 mm in length, thus the port 102 is at least 445 mm from the deepest part of the groove 106 in the shelf 110 to the bottom “floor” 108 to accommodate a 330 mm shaft length instrument.

Each port 102 has a throat that swallows a rotation wheel of the hand instrument such that the rotation wheel falls below the top opening when being inserted into the port 102. The throat is fractionally larger (e.g., 10% to 20% larger) in cross section than the rotation wheel such that the rotation wheel is held (restrained) laterally by upper walls (“throat walls”) of the port 102 when in the holder 100, e.g., as shown in FIGS. 6 and 7, the throat can have a back-to-front width between 7.5 cm and 4 cm, and as shown in FIG. 2A, a side-to-side width of between 3.5 cm and 5 cm (or 40 mm and 43 mm). The throat is configured to hold wheels with radii of substantially 40 to 50 mm. Rotation wheels can vary depending on manufacturer design, being 40 mm to 60 mm long (measuring parallel with the shaft) and having a radius of the wheel itself substantially 40 mm to 50 mm. Each port 102 has a substantially rectangular cross section, at least at the upper portion, such that it spans further from the patient (back to front) than parallel to a longitudinal axis of the patient (side to side) when in use. However, towards the bottom 108 of the port 102, the cross section may be substantially square or rectangular with side lengths of between 3.5 cm and 5 cm, or 40 mm and 43 mm, or 10 mm and 15 mm (within 10 mm of the bottom 108).

As shown in FIG. 1 and FIG. 8, the holder includes side walls 116B, 104A, 104B, 116A that form the left and right sides of the ports 102, and these side walls 116B, 104A, 104B, 116A are substantially vertical when in use. As shown in FIG. 8, the side walls 116A,B can have thicknesses of between 1 and 3 mm, e.g., substantially 2 mm or substantially 1.5 mm. Side wall 116B may be referred to as the left-side end wall.

In some embodiments, the left-side end wall 116B and/or the right-end side wall 116A may have linear/straight top edges, as shown in FIG. 1. In other embodiments, at least one of the instrument ports may include two downward grooves configured to receive and hold a handle of a laparoscopic hand instrument resting in the port, including the downward groove in a front wall and a further downward groove in a side wall of the port. In these embodiments, the top edges of the left-side end wall 116B and/or the right-end side wall 116A may include the further downward groove (“dip” or “gutter” or “notch”) configured to receive and hold a handle of a laparoscopic hand instrument resting in the corresponding port 102. Thus the end ports 102A,102C may each include two grooves: the groove 106 to the front, and the dip in the corresponding side wall 116A,B to the side. The dip in the side wall 116A,B may be substantially 65 mm wide and substantially 20 mm deep, and may have a V or U shape with a centre line and a pair of upward sloping walls that slope upward on both sides of the centre line. The dip in the left-side end wall 116B and/or the right-end side wall 116A serves as an additional groove to hold instrument handles, and allows larger instrument handles to hang out the sides of the holder instead of encroaching on neighbouring instruments sitting in the one or more centre instrument ports 102B. The dip in the left-side end wall 116B may orient the handle towards a left hand of the surgeon during use. The dip in the right-side end wall 116A may orient the handle towards a right hand of the surgeon during use.

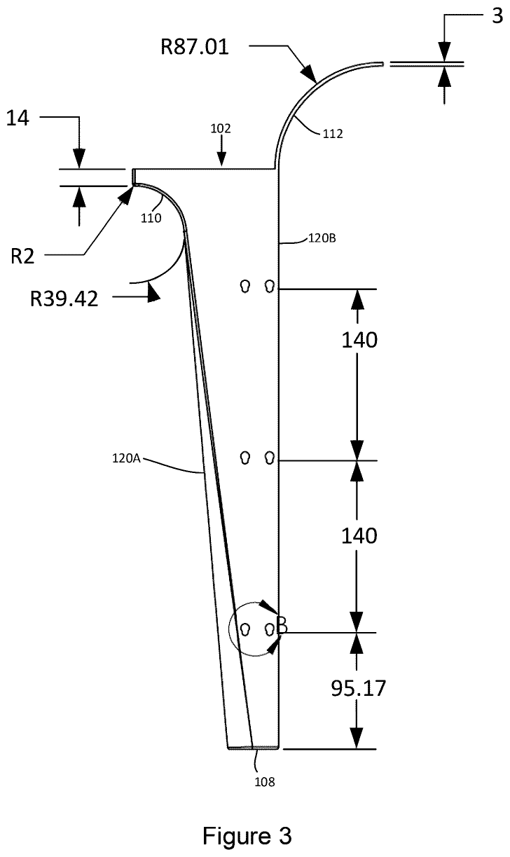

As shown in FIGS. 1 and 3, the holder includes a front wall 120A and a back wall 120B that form the front and back sides of the ports 102. As shown in FIGS. 3 and 7, the back wall 120B is substantially vertical when in use, and the front wall 120A is partially off vertical, between 0 degrees and 12 degrees (e.g., between 85 and 86 degrees as shown in FIG. 7), such that the ports 102 taper inwardly from their top openings to their bottoms 108.

The top opening of each instrument port 102 includes at least one angled guide to guide a distal end of the instrument (i.e., the end with the tip) into a throat of the port 102 when the instrument is being inserted into the port 102 (during use). The at least one angled guide provides an inward tapering opening to each port 102, making it easier for the surgeon to manually insert the instrument into the port 102, which may improve operation of the holder 100 when the surgeon is unable to carefully and accurately guide the instrument directly into the port 102, and/or allowing the port 102 to have a smaller throat while still having an easy-to-access mouth.

The at least one angled guide may include:
a. the shelf 110 (or “deck” or “platform”) projecting from the top opening towards the surgeon when in use (in a first direction); and/or
b. a leg curve 112 projecting from the top opening away from the surgeon when in use (in a second direction that is opposite to the first direction) and around/over/towards a leg of a patient.

As shown in FIG. 1, each port 102A, 102B and 102C has a corresponding shelf 110A, 110B and 110C, each of which slopes downwards into the corresponding port 102A, 102B and 102C. In some embodiments, the angled guides include both the shelf 110 and the leg curve 112, whereas in some other embodiments, the only angled guide is the shelf 110, i.e., the leg curve 112 need not always be required.

As shown in FIG. 7, the shelf 110 substantially extends the front-to-back width of the opening, effectively enlarging the top opening by at least 50%, 60% (e.g., from substantially 75 mm shown in FIG. 6 to substantially 120 mm as shown in FIG. 7), 70%, 80%, 100% (i.e., doubling the opening width), 150% or 200% (e.g., from substantially 40 mm, which would hold the rotation wheel in the throat, to substantially 120 mm as shown in FIG. 7, i.e., tripling the opening width).

The shelf 110 may include a substantially straight taper or slide into the top opening. Alternatively or additionally, as shown in FIGS. 3, the shelf 110 may include a shelf curve that curves into the top opening with increasing slope, e.g., substantially following a radius: this shelf radius may be between 3 cm and 5 cm, and the shelf curve may extend around substantially a quarter of a circle with the shelf radius. The shelf 110 may project at least partially from the top opening towards the surgeon at an angle of substantially 120 degrees from the longitudinal direction of the port 102, thus extending along the instrument handle when the instrument shaft is in the port 102 (when the shaft of the instrument is at 120 degrees to the longitudinal axis of the handle in the hand instrument).

As shown in FIG. 1, the shelf 110 includes the groove 106, so the groove 106 not only urges the handle into a centre orientation of the port 102 but also urges/guides the instrument into the port 102 during insertion.

As shown in FIG. 1, the leg curve 112 is on the other side of the port 102 from the shelf 110 and cooperates with the shelf 100 to provide the angled guide.

The leg curve 112 also keeps the holder 100 steady because the leg curve 112 rests at least partially on the patient (specifically on their leg), so the holder 100 thus rests at least partially on, or at least against, the patient leg (specifically upper leg or thigh) rather than just hanging from a sterile drape (also referred to as a “surgical drape”) that is placed over an upper leg of a patient, thus potentially improving the stability of the holder 100 when in use.

As shown in FIG. 1, the leg curve 112 includes a plurality of fastening points (which may be in the form of holes 114) configured to be attached to the patient via the sterile drape (which is itself fastened over the patient). The plurality of fastening points resist rotation of the holder 100 (including at least left-right rotation when fastened in place), and resist dislodgement. The fastening points include the holes 114 through the leg curve 112 that are configured to receive clips (e.g., towel clips or surgical clips) to fasten the leg curve 112 to the sterile drape.

As shown in FIG. 8, the holes 114 include two holes 114A and 114B, arranged substantially close to the left edge 122A and right edge 122B of the leg curve 112, e.g., between about 1 and 2 cm from the edges 122A, 122B respectively. The holes 114 may have average diameters of between 1 cm and 2 cm (or 15 mm or 17 mm).

As shown in FIG. 3, the leg curve 112 includes a curvature that substantially matches a curvature of an adult thigh, e.g., an average adult thigh or a large adult thigh, e.g., typical for a bariatric or gynaecological surgery patient. The curvature may include a radius of between 4 cm and 20 cm, including between 8 cm and 9 cm. The leg curve 112 may project from the top opening, and/or from a substantially straight back wall 120B of the ports, as shown in FIG. 3, around at least one quarter of a circle, or at least one eighth of a circle, or at least one sixteenth of a circle. Having a larger leg curve 112 can increase the stability and support is provides to the holder 100 during use in surgery, whereas having a smaller leg curve 112 can make it easier to stack and store during transport and storage before use in surgery.

The leg curve 112 is rigid, including formed of hard plastic and/or another rigid material, with a thickness of 1 mm to 5 mm, or 2 mm to 4 mm.

The holder 100 may have wall thicknesses of 1 mm to 3 mm. The holder 100, including the one or more instrument ports and the leg curve, may be formed of disposable materials. The holder 100, including the one or more instrument ports and the leg curve, may be formed of one or more injection molded polymers, forming a one-piece injection molded holder.

The holder 100 may include an attachable D-shaped port (“D port”) 400 configured to hold a laparoscopic energy instrument or suction/irrigation device with a power cord/cable and/or pipe/hose during surgery.

The D port 402 and an end (terminal) one of the instrument ports (102C) are configured to connect at least at their top openings to resist a surgeon manually inserting a laparoscopic instrument between the D port and the end during use (i.e., the D port 402 is fastenable to the hand instrument ports 102A, 102B, 102C via a connection to the terminal port 102C).

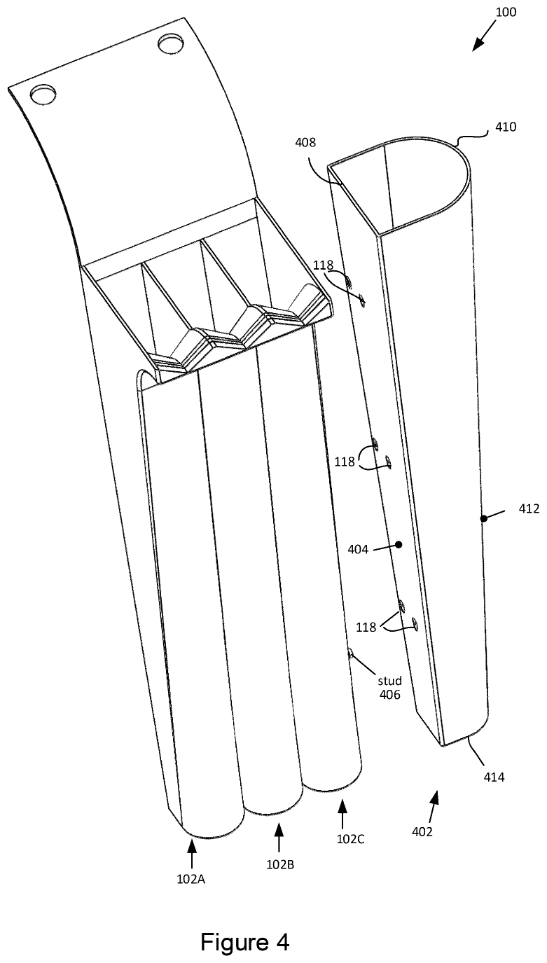

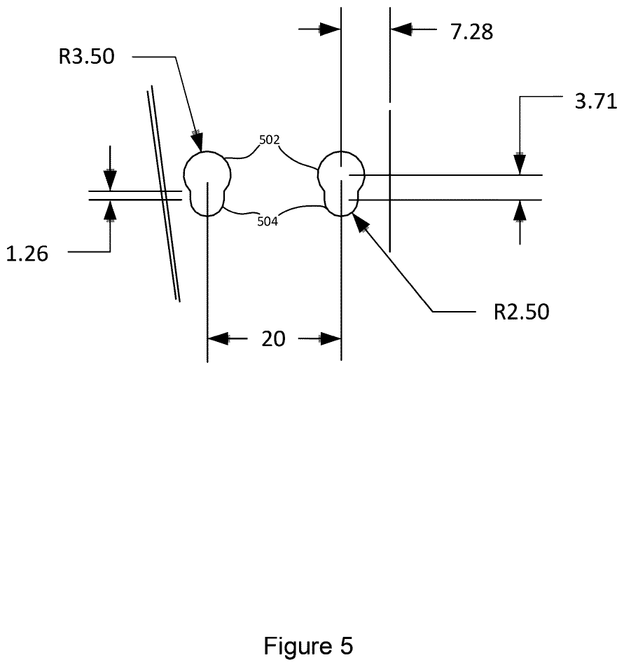

The terminal port 102C has a right-side end (terminal) wall 116A, e.g., as shown in FIG. 1, which may optionally be shaped to receive a cooperating wall 404 of the D port 402 to align the D port 402 adjacent to the terminal port 102C. As shown in FIGS. 1 and 4, the end wall 116A may be substantially flat and the cooperating wall 404 may be correspondingly and substantially flat. The D port 402 and the terminal port 102C may optionally include cooperating fastening elements to fasten the D port 402 to the terminal port 102C. The cooperating fastening elements (also known as “D part attachments”) may include slots 118 in the end wall, as shown in FIG. 1, configured (i.e., sized and located, e.g., in two vertical rows of three slots each) to receive cooperating projections 406 (“studs”) of the D port 402. Alternatively and/or additionally, as shown in FIG. 4, the cooperating fastening elements may include slots 118 formed in the D port 402 to receive cooperating studs 406 (at the locations of the slots 118 in FIG. 1) formed in the end wall 116A. As shown in FIG. 5, the slots 118 may include respective wide upper holes 502 and narrow lower holes 504, and the cooperating projections may include stems that fit through the narrow lower holes 504 and heads that fit through the wide upper holes 502 but not the narrow lower holes 504, thus sliding easily into the slots when the walls 116A and 404 are manually aligned and brought together, and then down (as the D port 402 hangs down by gravity from the terminal port 102C which itself is supported by the leg curve 112 or other means) such that the heads are secured by the narrow lower holes 504 thus keeping the D port 402 held against the terminal port 102C during use in surgery (but allowing manual removal of the D port 402 from the hand ports 102 after use if required, e.g., for cleaning and/or disposal). As shown in FIG. 5, the wide upper holes 502 can have an average radius of substantially 3 mm to 4 mm, and the narrow lower holes 504 can have an average radius of substantially 2 mm to 3 mm, and the respective centres of the wide upper holes 502 and the narrow lower holes 504 in each pair can be spaced by substantially 3 mm to 4 mm. The stud heads can have a diameter of substantially 6 mm.

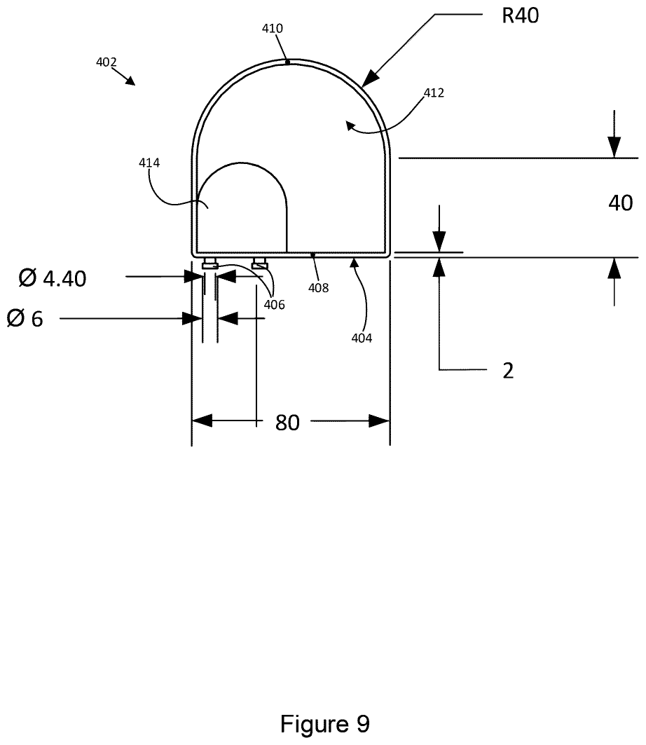

As shown in FIG. 4, the top opening of the D port 402 includes: a substantially straight edge 408; and a substantially curved edge 410 that cooperates with the straight edge 408 to form a “D” shape. Unlike the hand instrument ports 102, the D port 402 has a substantially flat top opening to support the handle of the laparoscopic energy instrument or suction/irrigation device while allowing the handle of the laparoscopic energy instrument or suction/irrigation device to rotate around the curved edge 410, e.g., as the power cord/cable and/or pipe/hose is moved during surgery (i.e., the curved edge 410 has no groove, so the energy instrument or suction/irrigation device is free to move). In other words, the D port 402 has a flat side 404 for facing and connecting to the terminal port 102C (and thus the other mutually connected ports 102), and an opposite curved side 412 that forms a D-shaped horizontal cross section of the D port 402. As shown in FIG. 9, the curved edge can have an average radius between 3 cm and 5 cm, or substantially 4 cm. As shown in FIG. 9, the wall thickness of the flat side 404 can be between 1 mm and 3 mm, or substantially 2 mm. The wall thickness of the curved side 412 can also be between 1 mm and 3 mm, or substantially 2 mm. As shown in FIG. 9, a D bottom 414 of the D port 402 can have a D-shaped bottom plane, mirroring the D shape of the top opening, albeit smaller (e.g., with an average radius between 1 cm and 3 cm, or substantially 2 cm, such as half the radius of the top opening), and aligned to connect to the bottom 108 of the hand ports 102 (specifically the bottom 108C of the terminal port 102C) such that the flat side 404 hangs against the end wall 116A of the hand ports 102 as described hereinbefore.

The energy instruments held in the D port 402 may include elongated electrocautery instruments that are commonly used in laparoscopic operations and have extremely high temperatures and need to be stored in a position that prevents contact with the patient and/or flammable materials, e.g., the drape. These instruments have an electrical cable that needs to be kept away from the other surgical instruments to prevent entanglement.

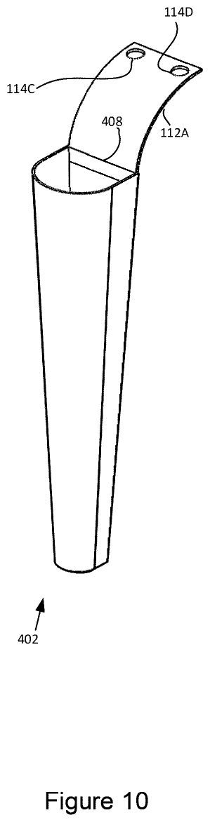

As shown in FIG. 10, the D port 402 may, in some implementations, include a D-port portion 112A of the leg curve (or “D-port leg curve”) with the substantially same length, thickness and radius as the leg curve 112 (i.e., substantially the same dimensions except the left-right width may be less depending on the size of the “D” shape). As shown in FIG. 10, the D-port portion 112A includes a plurality of fastening points for fastening to the surgical drape—these fastening points may include holes 114C and 114D with substantially the same dimensions as the holes 114A and 114B. The D port 402 may include the D-port leg curve 112A on its wall 404 instead of the include cooperating fastening elements, as shown in FIG. 10, so the D port 402 can be hung adjacent to the terminal port 102C on the surgical drape using clips though the holes 114C and 114D. The apparatus 100 may thus include the D port 402 configured to hold one laparoscopic instrument during surgery; and the leg curve 112A projecting from a top openings of the D port away from the surgeon when in use and round/over/towards the leg of the patient to rest partially on the upper leg of the patient.

As shown in FIG. 4, the substantially straight edge 408 may be configured and used for resting adjacent the terminal port 102C. Alternatively, as shown in FIG. 10, the substantially straight edge 408 may be attached to and form part of the leg curve 112A.

Each hand instrument port 102 may include a window of transparent, waterproof material configured to display a distal end of the instrument in the port (including a tip of the instrument) to a surgeon (to allow the surgeon to see the tip of the instrument when the instrument is resting in the port). As typical hand instruments have handles and tips that are interchangeable, it can be hard to distinguish a tip type based on a handle: thus the hand instruments can all look the same when stored although have different tips that perform different functions. The window allows the tips of the instruments to be identified whilst in the holder 100.

The window may include transparent, waterproof walls of the ports 102. The window portion may include clear plastic material.

The window may extend from the bottom 108 of the ports 102, on the same side as the deck/shelf 110 so the surgeon can see the instrument tips during use. The window may extend across all ports 102 and be at least 50 mm to 100 mm high from the bottoms 108. Alternatively, at least the front of each port 102 can be transparent to provide the window. The window may include a transparent polymer, e.g., Polypropylene (PP) or polycarbonate.

The holder 100 may include 2 ports, and may be configured to be assembled with another holder 100 using slots and studs with the same configurations as the slots 118 and studs 406 described hereinbefore.

### IMPLEMENTATIONS

The holder described herein may mitigate problems with cylindrical instrument holders (“laparoscopic quivers”) in which the instruments are stored in random orientations, making access and swapping of the instruments difficult, and resulting in entanglement of the handles and/or cables/hoses of energy instruments or suction/irrigation devices within the cylindrical holder.

The holder described herein may mitigate problems with opaque instrument holders, e.g., made from opaque plastic. The opaque nature of such holders prevents visualisation of the individual instrument tips. Given that the grasping mechanisms all look the same it can be difficult to identify for a surgeon/nurse to identify which instrument is required.

The holder described herein may mitigate problems with cylindrical or largely rigid, straight holders that are configured to be secured to the sterile drape at only one point. Such a rigid cylindrical holder does not easily rest on the patient's upper leg, which is generally curved, and so dislodgement is common, resulting in instruments falling to the operating room floor.

### INTERPRETATION

Many modifications will be apparent to those skilled in the art without departing from the scope of the present invention.

The presence of “/” in a FIG. or text herein is understood to mean “and/or” unless otherwise indicated. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “substantially” can indicate a percentage greater than or equal to 80% or 90%, for instance, 92.5%, 95%, 97.5%, 99%, or 100%.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavor to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. An apparatus including:
one or more instrument ports configured to hold respectively one or more laparoscopic instruments during surgery,
wherein a top opening of each instrument port of the one or more instrument ports includes a downward groove configured to receive and hold a handle of a laparoscopic hand instrument resting in the instrument port such that the handle is oriented towards a surgeon during use.

2. The apparatus of claim 1, wherein the one or more instrument ports includes a plurality of ports, and the downward grooves of adjacent ports are oriented around their respective top openings such that handles of adjacent hand instruments, when in the adjacent ports, are guided away from overlapping each other.

3. The apparatus of claim 1, wherein the downward groove includes at least two edges having opposite slopes meeting at a low point or line of the downward groove when in use such that the handle is urged towards a low point/line by gravity, thus orienting the handle toward the surgeon.

4. The apparatus of claim 3, wherein the downward groove is a V groove formed of two straight edges or a U groove formed of curved edges; and/or
wherein at least one of the one or more instrument ports includes two downward grooves configured to receive and hold the handle of the laparoscopic hand instrument resting in the at least one of the one or more instrument ports, including the downward groove in a front wall and a further downward groove in a side wall of the at least one of the one or more instrument ports.

5. The apparatus of claim 1, wherein each instrument port of the one or more instrument ports includes walls, including lower walls formed of water-proof material and a rigid material, e.g., a hard plastic,
wherein each instrument port of the one or more instrument ports includes a lower portion that is enclosed by the lower walls to hold a distal end of the laparoscopic hand instrument including a tip in a sterile manner,
wherein the lower portion includes a sealed bottom, and/or
wherein each instrument port of the one or more instrument ports has a length, extending from its top opening to its sealed bottom, that is configured to hold hand instruments with lengths of substantially 250 mm, 330 mm and/or 430 mm.

6. The apparatus of claim 1, wherein each instrument port of the one or more instrument ports has a throat that swallows a rotation wheel of the laparoscopic hand instrument such that the rotation wheel falls below the top opening when being inserted into the instrument port, and/or
wherein the throat is fractionally larger in cross section than the rotation wheel such that the rotation wheel is held laterally by upper walls of the instrument port when in a holder, including for wheels with radii of substantially 40 to 50 mm.

7. The apparatus of claim 1, wherein each instrument port of the one or more instrument ports has a rectangular cross section such that it spans further from a patient than parallel to a longitudinal axis of the patient when in use, and/or
wherein walls of the one or more instrument ports are substantially vertical when in use, including an inward taper from the top opening to a bottom.

8. The apparatus of claim 1, wherein the one or more instrument ports include disposable materials,
wherein the one or more instrument ports include injection moulded plastic, and/or
wherein the one or more instrument ports include 2, 3, 4 or 5 hand instrument ports.

9. The apparatus of claim 1, wherein the top opening of each instrument port of the one or more instrument ports includes at least one angled guide to guide a distal end of the laparoscopic hand instrument into a throat of the instrument port when the laparoscopic hand instrument is being inserted into the instrument port during use.

10. The apparatus of claim 9, wherein the at least one angled guide provides an inward tapering opening to the each instrument port of the one or more instrument ports, making it easier for the surgeon to manually insert the laparoscopic hand instrument into the instrument port.

11. The apparatus of claim 9, wherein the at least one angled guide includes:
a shelf projecting from the top opening towards the surgeon when in use in a first direction; and/or
a leg curve projecting from the top opening away from the surgeon when in use in a second direction and around/over/towards a leg of a patient.

12. The apparatus of claim 11, wherein the shelf includes the downward groove of each port.

13. The apparatus of claim 1, including: a leg curve projecting from top openings of the one or more instrument ports away from the surgeon when in use and around/over/towards a leg of a patient to rest partially on an upper leg of the patient.

14. An apparatus including:
one or more instrument ports configured to hold respectively one or more laparoscopic instruments during surgery; and
a leg curve projecting from top openings of the one or more instrument ports away from a surgeon when in use and around/over/towards a leg of a patient to rest partially on an upper leg of the patient.

15. The apparatus of claim 14, wherein the leg curve includes a plurality of fastening points configured to be attached to the patient, including to a sterile drape fastened over the patient.

16. The apparatus of claim 15, wherein the plurality of fastening points include holes through the leg curve that are configured to receive clips to fasten the leg curve to the sterile drape.

17. The apparatus of claim 14, wherein the leg curve includes a curvature that substantially matches a curvature of an adult thigh.

18.-26. (canceled)

27. The apparatus of claim 1, wherein the one or more instrument ports includes a plurality of ports, and wherein adjacent ones of the plurality of ports are connected at least at their top openings to resist the surgeon manually inserting a laparoscopic instrument between the adjacent ones of the plurality of ports during use.

28. The apparatus of claim 27, wherein the adjacent ones of the one or more instrument ports form one or more pairs of adjacent ports, and the adjacent ports in each pair of the one or more pairs of adjacent ports are separated by a separating wall (“dividing wall”) that provides a wall for both of the adjacent ports.

29. The apparatus of claim 1, wherein each instrument port of the one or more instrument ports includes a window of transparent, waterproof material configured to display a distal end of the laparoscopic hand instrument in the instrument port, including a tip of the laparoscopic hand instrument, to the surgeon to allow the surgeon to see the tip of the laparoscopic hand instrument when the laparoscopic hand instrument is resting in the instrument port.

30.-31. (canceled)
